(19) United States
(12) Patent Application Publication
(10) Pub. No.: US 2016/0367261 A1
(43) Pub. Date: Dec. 22, 2016

(54) Local Embolization Using Thermosensitive Polymers

(71) Applicant: Genzyme Corporation, Cambridge, MA (US)

(72) Inventors: Jean-Marie Vogel, Cambridge, MA (US); John A. Merhige, Sudbury, MA (US)

(21) Appl. No.: 15/002,113

(22) Filed: Jan. 20, 2016

Related U.S. Application Data
(63) Continuation of application No. 12/920,052, filed on Dec. 30, 2010, now abandoned.
(60) Provisional application No. 61/032,555, filed on Feb. 29, 2008.

(51) Int. Cl. A61B 17/12; A61L 24/00; A61L 24/04; A61B 18/04

(52) U.S. Cl. CPC A61B 17/12181; A61B 18/04; A61L 24/001; A61L 24/046; A61L 24/0015; A61L 2430/36

(57) ABSTRACT

Precision in thermotherapy is obtained by providing a reverse gelling polymer composition which gels when its temperature is raised towards body temperature. The composition is injected into the blood supply of the tissue being treated, at the beginning of thermotherapy. The temperature increase caused by the heating rapidly gels the composition, which temporarily blocks the flow of blood in the region being treated. This improves the predictability and stability of treatment. On cessation of heating, the composition gradually dissolves, removing the temporary embolization. The use of local heating can also expedite removal of tumors and the like from soft organs, even when the heating itself has no therapeutic effect.

### RELATED APPLICATIONS

This application is a continuation application of U.S. application Ser. No. 12/920,052, filed on Feb. 19, 2009, which is the U.S. National Stage of International Application No. PCT/US2009/034479, filed on Feb. 19, 2009, published in English, which claims the benefit of U.S. Provisional Application No. 61/032,555, filed on Feb. 29, 2008. The entire teachings of the above application(s) are incorporated herein by reference.

### BACKGROUND OF THE INVENTION

Thermotherapy is a promising approach to the precise and selective removal of internal tissue. In thermotherapy, a localized source of thermal energy, such as a radiofrequency (RF) or microwave emitting probe, is positioned within or next to a volume of tissue which should be removed. Positioning is typically obtained by minimally invasive methods, for example via a catheter in an artery or vein. Mild heat is then applied to the tissue, and surrounding cells are directly killed, or induced to enter apoptosis or otherwise induced to die. In some cases, for example when the access route is intravascular, a cooling flow is placed next to tissue that is to be preserved, such as the wall of the blood vessel itself. Thermotherapy is generally conducted at temperatures in the range of about 37 to 50° C., and is distinguished from higher-temperature treatments such as cautery.

One difficulty in such methods is controlling for the effect of blood flow within the tissue on the desired temperature pattern. Generally, blood flow will remove heat from tissues being treated, and carry it downstream to tissues whose treatment is not intended. Because the pattern of blood flow on a small scale is not well determined, the effect of the blood flow cannot be accurately compensated for, and so some tissue that should be ablated may survive. Such a complication is especially undesirable if the tissue being treated is metastatic.

One approach to overcoming these difficulties is described in our co-pending application “Perfusive Organ Hemostasis”, U.S. 60/874,062 (incorporated herein by reference). In this approach, the organ is perfused with a reverse-gelling polymer, of a composition and at a concentration selected so that the gelling temperature Tg is somewhat below body temperature, so that the polymer solution gels as its temperature rises towards body temperature. Then, when flow of the polymer into the organ slows or ceases, the polymer gels as it reaches body temperature. As shown in US publication 2005/0008610 (incorporated herein by reference), this procedure can be used to temporarily embolize an artery, and U.S. 60/874,062 (incorporated herein by reference) shows a first method of application for providing hemostasis in an entire organ. The reverse gelling polymer, such as certain poloxamers, will gradually dissolve in the blood as individual molecules diffuse away from the gelled region, and as serum diffuses into the gel. As a result, the gel eventually liquefies. The time to liquefy can be controlled by a combination of selection of the chemical composition of the polymer, the concentration of the polymer in the solution applied, the purity of the polymer, and the amount of solution applied.

However, in large organs, for example the liver, the amount of polymer composition required to form a gel can be large. While many reverse gelling polymers are known to be safe in the mammalian body in reasonable amounts, the volume administered should be minimized. Moreover, in a large organ, it can be difficult to determine an appropriate site from which to embolize a small area, since branching patterns of veins and arteries on smaller scales are often non-standard. Hence, a better method of local temporary embolization would be useful in surgery, especially in surgery of large and/or highly vascularized organs.

### SUMMARY OF THE INVENTION

The present invention describes an improved method for temporary embolization of an organ or a region thereof, to facilitate the performance of a surgical or medical procedure at a site in the organ. In a first embodiment, an embolizing solution is provided that comprises a reverse-gelling polymer composition that gels as the temperature rises towards body temperature. The organ, or a region of the organ, is perfused with an embolizing solution comprising this polymer. The particular polymer and its concentration are selected so that gelation is slow at temperatures significantly below local body temperature. Then, before and during perfusion, the temperature is elevated in a site of the organ in which hemostasis is desired. As a result, the polymer solution gels rapidly at the intended site, and slowly away from the site. The region of embolization thus tends to be restricted to the actual site of operation, instead of most or all of the organ containing the site of interest.

This increase in temperature may be accomplished by any convenient means, for example by the induction of heating by the application of RF (radio frequency) energy, or by heating via optical energy transfer from visible or infrared light, or by other local heating means, such as applying a heated liquid or gas, or by heat transfer from a solid object. In one embodiment, the heating in question is also a heating administered for therapeutic purposes, such as tissue ablation. The elevated temperature at the site causes the reverse gelling polymer composition (RGP) to gel, thereby locally embolizing the site and achieving reversible hemostasis. Administration of RGP is typically discontinued once temporary local hemostasis is achieved.

Once local hemostasis is achieved at the selected site, the surgical or medical procedure is initiated or continued. For example, more intense RF energy could be used to destroy a tumor, or a low-energy field can be used for a selected time to kill cells or induce apoptosis. After performing any required suturing, reinforcing or other repair procedure, the low-intensity heating field is removed, resulting in the prompt cooling of the affected tissue to body temperature. The selected polymer solution is still gelled at body temperature, but because it is near its critical gelling concentration, the dilution of the RGP that occurs by diffusion of individual polymer molecules away from the gelled site causes the local hemostasis to be rapidly released. Optionally, additional rapid local cooling can be achieved by perfusion of unblocked circulation within the organ, and optionally the organ's exterior, with cold isotonic solutions, further accelerating the return of normal circulation.

In another embodiment, the heating of the tissue is provided primarily or entirely for the induction of temporary hemostasis by a reversible embolization of the tissue with a reverse gelling polymer solution that has a gelling temperature Tg that is below normal body temperature. While the site is embolized, a portion of the tissue is removed by standard surgical means. The site of removal is then treated to prevent bleeding or other fluid efflux, for example by suturing, cautery, application of sealing materials, application of reinforcing materials, and other conventional methods of surgical practice. Then the heating is discontinued and the tissue is allowed to return to normal body temperature, optionally accelerated by application of cold fluids to the site. As polymer molecules diffuse away from the gelled region, the embolization gradually dissipates. The rate of dissolution can be accelerated by chilling the site below body temperature, because at temperatures below Tg, the gel will convert back into a solution, ending the hemostasis.

This improved procedure thus gives the physician significantly more control over the timing of reperfusion in such operations. Moreover, even in the gelled state, the gelled polymer will gradually dissolve in the surrounding tissue, and in any blood it is in contact with, therefore reliably removing hemostasis in a reasonably predictable interval. The ability to remove unwanted tissue first and then cauterize or otherwise seal it can be advantageous in minimizing the collateral damage to the organ.

In certain embodiments, the invention comprises a method of producing temporary hemostasis in a site in the tissue of a mammal, the method comprising the steps of:

a) introducing into the vasculature of said tissue, at a location leading through the circulation to said site, a temporary embolizing solution comprising a reverse thermosensitive polymer, wherein said embolizing solution has a composition and a concentration which causes it to gel sufficiently at a gel temperature Tg to effectively stop blood flow at said site, said temperature Tg being below the local temperature of the tissue being treated; b) perfusing said site with said reverse thermosensitive polymer composition; and c) before or during said perfusion, heating said site to a temperature above local body temperature; thereby producing temporary hemostasis at said site of said mammal.

In this method, the gel temperature Tg of said embolizing solution is between about 30° C. and about 34° C. The site is temporarily embolized by perfusing a larger region of tissue in which said site is located with said embolizing solution, but heating only near the site, thereby initially forming a gel only in the vicinity of said site. Once a gel has formed at a site, perfusion of polymer is stopped. For example, a bolus of polymer solution that is sufficient, based on experience, to embolize a heated site is supplied, and no further administration of polymer solution is made until the presence or absence of hemostasis is observed.

The local tissue temperature, before heating of the site, will be 37° C. in most cases, but may be lower. The reverse thermosensitive polymer or copolymer is typically a block copolymer, but may be a random copolymer, graft copolymer, or branched polymer or copolymer. In a preferred embodiment, the reverse thermosensitive polymer is a block copolymer, such as a polyoxyalkylene block copolymer, optionally with some amine connecting groups, and in a more preferred embodiment is a poloxamer or poloxamine. For example, the reverse thermosensitive polymer may be one or more of poloxamers 407, 188, 118, 338, F127 and F108, or poloxamines 1107 and 1307. Other poloxamers, and equivalent poloxamers from other manufacturers, may be used. The reverse thermosensitive polymer is preferably a fractionated or purified poloxamer or poloxamine, prepared by known literature methods. Suitable methods may be found in, for example, U.S. Pat. No. 6,761,824 B2, U.S. Pat. No. 6,977,045 B2; and U.S. Pat. No. 5,800,711. The elimination of contaminants has been found, as described in these references, to narrow the temperature range in which the polymer solution converts from a liquid to a gel.

In the application of the method, perfusion preferably begins after the beginning of said heating. The heating of the organ is provided by one or more of electromagnetic radiation, sonic energy, heated fluid, a heating pad, a heating element, and heat produced by a surgical tool or instrument. In particular, the heating of the organ is provided by electromagnetic radiation.

In another embodiment, a method for performing a surgical procedure at a site in a tissue of a mammal may comprise the steps of accessing the vasculature providing blood to said site, upstream of said site, with a fluid delivery system; delivering through said fluid delivery system an embolizing solution comprising a reverse gelling polymer that gels when its temperature rises to a temperature Tg which is below normal local tissue temperature; warming said embolizing solution above local tissue temperature at or near said site, thereby gelling the embolizing solution to embolize said site; maintaining said warming throughout the performance of the surgical procedure, thereby maintaining hemostasis at the site; and discontinuing the heating at the close of the procedure, thereby allowing the polymer to diffuse away from the gel, thereby releasing hemostasis and allowing resumption of blood flow at the site.

The embolizing solution that gels below local tissue temperature preferably comprises one or more poloxamers or poloxamines as reverse gelling polymer. The warming of the solution may be at least in part due to warming of the tissue by the process of performing the procedure. The process of performing the procedure may include the use of RF (radiofrequency) energy to remove, treat or cauterize tissue. The site of the procedure will most commonly be in a tissue selected from liver, uterus, prostate, brain, spleen, pancreas, gall bladder, lung, breast, and kidney, without excluding other sites of use. The treatment may be for the removal or cure of a cancer, a benign tumor or growth, or a hemorrhage.

The embolizing solution comprising a reverse thermosensitive polymer may further comprise a contrast-enhancing agent, which may be selected from the group consisting of radiopaque materials, paramagnetic materials, heavy atoms, transition metals, lanthanides, actinides, dyes, and radionuclide-containing materials. The embolizing solution may further comprise a biologically active agent, for example but without limitation selected from anti-inflammatories, antibiotics, antimicrobials, antivirals, analgesics, antiproliferatives, and chemotherapeutics.

In any of these versions of the method, the site may be closed with at least one of sutures, staples, sealant, adhesive, and hemostatic agent, before the reduction of temperature to allow reperfusion of the organ by blood. Moreover, after completion of the procedure, the reperfusion of the organ may be accelerated by circulation of isotonic fluid at a temperature of less than 37° C. by one or more route selected from a route that passes through the organ and a route that passes along the exterior of the organ. The temperature of the reperfusing fluid may be less than 30° C.

In another aspect, the efficacy of thermotherapeutic treatment of tissues is improved by a method comprising using a thermotherapeutic device create to heat at a site to be treated; perfusing the site with an embolizing composition comprising a reverse gelling polymer, said polymer characterized in gelling sufficiently at a temperature below body temperature to produce local hemostasis; and treating the site by thermotherapy in a conventional manner. In this method, the perfusion with the embolizing solution containing a reverse gelling polymer produces at least one of a more reliable and a more predictable extent of tissue treatment, than occurs without the use of said reverse gelling composition.

The invention also comprises a system for thermal treatment of an organ, the system comprising means for applying heat to a localized region of an organ, to selectively destroy tissue by heating it to a temperature above 37° C. and below a maximum temperature of about 50° C.; means for locally perfusing said localized region of an organ with an embolizing solution comprising a reverse gelling polymer, wherein the gelling temperature Tg for said reverse gelling polymer is below 37° C.; and whereby reversible local hemostasis is obtained at the site of thermal treatment while heat is applied to said localized region, and said hemostasis spontaneously ceases after the application of said thermal treatment ceases, due to one or more of diffusion of polymer molecules away from the gel, and liquefaction of the gel due to cooling of the tissue below Tg.

In another aspect, the invention comprises a medicament for improving the outcome of surgery by temporarily embolizing a site at which surgery is conducted, the medicament comprising a reverse gelling polymer infused into an organ said site, wherein the medicament is temporarily immobilized at said site by local tissue heating before it is immobilized at locations remote from said heated site.

In another aspect, the invention comprises the use of a reverse-gelling polymeric solution to produce local reversible hemostasis at a site, wherein the reverse-gelling polymeric solution gels at a temperature below the body temperature at the site, and the gelation is made to occur more rapidly by the localized heating of the site at a temperature above the gelation temperature of the polymer solution.

In another aspect, the invention comprises the use of an embolizing solution to facilitate surgical removal of a selected part of an organ, wherein the use comprises the provision of an embolizing solution comprising a reverse-gelling polymer, initially at a temperature below its gelling temperature Tg, to at least said selected part of said organ while said organ is heated to a temperature above body temperature, whereby said reverse-gelling polymer is caused to gel sufficiently to produce hemostatis; and wherein while the organ is temporarily embolized, said selected part of said organ is removed by surgery, and then the remaining part of said organ is treated to seal its surface sufficiently to prevent loss of blood or other bodily fluids; and then, upon ceasing to heat said organ, the embolization is reversed because of the diffusion of polymer molecules out of the gel, thereby allowing blood flow in the remainder of said organ.

### BRIEF DESCRIPTION OF THE FIGURES

FIG. 1 schematically illustrates a thermotherapy treatment site, and shows deviations in areas of effective treatment due to blood flow.

### DETAILED DESCRIPTION OF THE INVENTION

The invention will now be described more fully with reference to the accompanying examples, in which certain preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Surgically removing only the morbid part of an internal organ, such as a kidney, or only a selected portion of hyperplastic tissue, as in benign prostate hyperplasia, can be beneficial for the patient in that at least part of the functionality of the organ can often be spared. However, many of the organs that might benefit the patient if only part of the organ is removed are soft, and/or prone to bleed extensively, and/or have differing compartments, whose contents should not be allowed to mix (e.g., the kidney or liver.) For example, essentially normal kidney function can be preserved with less than one-half of the normal functionality of one of the two kidneys, and the liver can regenerate if sufficient detoxification potential is retained or provided artificially. The challenge to the surgeon is to efficiently and completely close such organs, after removal of a tumor or other abnormality, so that blood does not leak into the abdominal cavity, and so that the separation functions of the organs can rapidly regenerate.

We have found, as published in patents and patent applications, that the use of a reverse-gelling polymer—i.e., a polymer that gels as the temperature rises above a certain temperature (Tg)—can temporarily embolize the arteries (US 2005/0008610, incorporated herein by reference) and other internal organs (Schwartz et al., U.S. 60/874,062, incorporated herein by reference); Raymond et al., Biomaterials 2004 vol. 25, p. 3983). Preliminary preclinical and clinical results appear promising.

However, there are some uncertainties in the procedure and areas that can be improved. One uncertainty that one would like to reduce is the length of time needed to reperfuse the organ, after surgery and any necessary sealing or suturing is complete. This is because when the circulation is blocked, the affected region becomes anoxic. For brief periods, the anoxia is largely reversible, but damage does accrue, and the ability to reverse the damage upon reperfusion declines with the time of anoxia, at a rate that is organ dependent. Hence, rapid reversal of the temporary embolization is highly desirable.

Application of cold solutions, such as cool or cold isotonic saline, will reverse the gelled state of the RGP, but it is not always feasible to do this quickly via the circulation itself, since the circulation is locally blocked by the reverse gelled polymer gel. Hence, reperfusion is dependent on a combination of external cooling, and gradual dilution of the gel by the diffusion of molecules from the gel into the upstream or downstream circulation, or into tissue interstitial spaces and the like.

Another problem to be addressed is the avoidance of hemostasis of an entire organ, when what is required is hemostasis in the vicinity of a particular site. If circulation can be maintained in those parts of the organ not requiring surgery, and if the volume of tissue subjected to hemostasis can be minimized, then outcome can be improved, and in particular the likelihood of the organ remaining at least partially functional at the end of the procedure is markedly improved.

Another problem to be addressed is to prevent the flow of blood, in an organ being treated by heat or radiant energy, from distorting the zone of treatment by carrying heat from tissue intended to be treated, to other tissue outside the treatment zone.

In response to these and other needs, a new approach to the problems of creating an embolized zone at the site of an operative procedure, and of removing an embolizing gel at the end of the procedure, and of maintaining perfusion in zones of the organ away from the operative site, has been invented. The new approach arises from the production of a reverse gelling polymer that gels over a relatively narrow range that is a few degrees below body temperature, for security of gelation, but which is applied at a low concentration, which is above the minimum concentration required for reverse thermal gelation, but otherwise is as low as is feasible. Using the most dilute polymer solution available, that will still gel at the selected temperature, optimizes the speed at which the gel will dissolve due to diffusion of polymer molecules away from the gelled zone. Gelation, and local embolization producing hemostasis, is then produced by replacing some or all of the blood in the organ with a reversible heat-gellable polymer solution, and the gelation of the polymer at the target site is enhanced and made more rapid and stable by local heating at the site. Where possible, the gellable polymer is only instilled into regions of the organ that are to be treated.

In particular, in the materials and procedures of the invention, the gelation temperature is lower than local body temperature. Body temperature is about 37° C. internally, and so gelling temperatures of the heat-gellable polymer solution, for internal use, should be in the range of 28° C. or preferably at least 30° C., up to about 36° C., more preferably in the range of about 30-35 deg. C., still more preferably in the range of about 31-34° C. If the polymer is to be used in or near the skin for a procedure, or otherwise in a body region where the overall temperature is below 37° C., the preferred reverse gelling temperature of the gel may be correspondingly lower, depending on the temperature to be induced in the particular tissue by the heating procedure.

### Examples of Polymers

It is known that in certain concentration ranges, the gelling temperature of a reverse gelling polymer changes as the polymer concentration is varied. Most commonly, the gelling temperature of a RGP polymer increases as the concentration is reduced, until the polymer fails to gel. Hence, it is possible to select gelling temperatures of RGP solutions by a combination of selection of a poloxamer or other RGP composition, and by selection of its concentration.

Poloxamers are preferred RGPs in the invention. Poloxamers are a well-known class of polyalkyleneoxide copolymers, typically composed of a core block of poly(propylene oxide) tipped at each terminus with a block of poly(ethylene oxide). Most commonly, the polymer is unbranched. Poloxamers having a higher proportion of propylene oxide tend to exhibit the reverse gelling phenomenon. The poloxamer solution is preferably fractionated to narrow the gelling range. Fractionation is described for example by Reeve et al., in U.S. Pat. No. 5,800,711, U.S. Pat. No. 6,761,824 and U.S. Pat. No. 6,977,045 (incorporated herein by reference). The fractionation procedure also tends to reduce the width of the temperature range over which viscosity rises rapidly with temperature, which simplifies the mechanical requirements, such as applied pressure, for administration of the polymer.

Poloxamers such as BASF poloxamers 407, 188, 118 and 338, and poloxamines such as 1107 and 1307, and “Pluronic” brand poloxamers, for example F127 and 108, may be suitable, after purification and selection of concentration, for use in 37° C. environments, or in colder environments near body surfaces. In use, the polymer is provided in a sterile solution of suitable salinity or tonicity for the task or procedure to be conducted. Poloxamines, in which amine groups replace oxygens in the backbone or ends, can also be used.

### Examples of Organs and Diseases of Interest

The methods of the invention can be used in any organ or situation in the body where temporary but completely reversible hemostasis is desired. The salient feature of the invention, as opposed to other inventions involving temporary hemostasis with reverse gelling polymers, is that the polymers in the present invention are selected to gel at temperatures somewhat below the local tissue temperature, and are controlled in concentration, so as to minimize the duration of hemostasis at body temperature. Then the region to be treated is raised in temperature to a temperature above body temperature. This partially stabilizes the polymer in the gelled state. At the conclusion of treatment, the heating is discontinued. Temperature drops rapidly to body temperature, which increases the rate of loss of polymer molecules from the gelled polymer.

The methods of the invention are particularly advantageous when used in conjunction with a therapeutic effect of the localized heating. The treatment in which the reverse gelling polymer is provided may be for any purpose, including without limitation treatment for the removal or cure of a cancer, a benign tumor or growth, or a hemorrhage. Any tissue may be involved, including without limitation liver, uterus, prostate, brain, spleen, pancreas, gall bladder, lung, breast, and kidney.

The local embolization of tissue and organs with reverse gelling polymers has been described elsewhere, for example in other patent applications by applicants (e.g., US 2005/0008610, incorporated herein by reference), for local embolization occurring without an ancillary heat source. Local embolization with reverse-gelling polymers gelling above body temperature is the subject of a copending application. A system not requiring local heating will generally be simpler when it is effective, and so will be preferred.

However, in some situations, the use of embolization with reverse-gelling polymers while heating the affected above body temperature is preferred, and has several advantages. First, a general advantage of the procedure is that it tends to minimize the amount of polymer temporarily deposited in the organ. Second, it tends to minimize the volume of tissue in which hemostasis is established, minimizing anoxia in tissues of the organ of interest and in surrounding tissues. Third, the re-liquefaction of the polymer at temperatures above body temperature leads to rapid cessation of hemostasis at the conclusion of the procedure. Fourth, the need for additional heating allows a more precise localization of the tissue region in which hemostasis is achieved.

### Routes of Heating

Any method of heating can be used. The heating of the organ can be provided by one or more of electromagnetic radiation, sonic energy, heated fluid, a heating pad, a heating element, and heat produced by a surgical tool or instrument. Suitable methods include, without limitation, the use of microwaves, radio-frequency waves, infrared and visible light, and other non-ionizing electromagnetic radiation. Electromagnetic radiation can be delivered to the exterior of a body or organ, or to interior sites via catheters, local generators, or the like. Direct heating can be used by contact of a heating unit with the exterior of a body or tissue, or via catheters or other internal probes. Heating of the target site can also be via electrical heating of a resistance, or by circulation of a heated fluid inside a device in contact with the tissue site. Heating can be accomplished by heating a natural fluid, particularly blood or a temporary substitute for blood that is placed into the circulation, that will circulate to the site. Heating can be accomplished by suspending the organ, or a region of the body, in a heated fluid, such as water, saline or the like. Heating can be achieved via ultrasound and other vibratory mechanisms.

### Degree of Heating

The primary function of heating the affected tissue is for therapeutic purposes, and this will determine the desired temperature rise at the site of treatment. A secondary function of the heating of the affected tissue is to stabilize a gel that is near the concentration limit for gelation, by increasing the temperature and thus making the polymer less soluble. As is shown in the Reeve et al. references above, a purified poloxamer will typically go from moderately viscous to effectively gelled over a range of about 3 to 5° C. Hence, a poloxamer solution that is still liquid at, for example, 30° C., will tend to gel in the region of 33-36° C. If the tissue at the selected site has a temperature of 37° C., then gelation will typically be slow. If the temperature is a few degrees higher, then gelation will be relatively quick, and the polymers will also be less soluble, and so less likely to diffuse away from the region of the gel.

Because such a polymer solution will gel more slowly at body temperature than in the heated region, it will tend to become localized in the heated region around the site of the procedure. Elsewhere, it will tend to gel slowly, and therefore to some extent will not gel at all, especially as it moves into the venous circulation and is increasingly diluted. This allows tissue in parts of the organ that are away from the site being operated on, to not be embolized. This reduces tissue damage and promotes recovery of organ function.

### Control of Heat Distribution

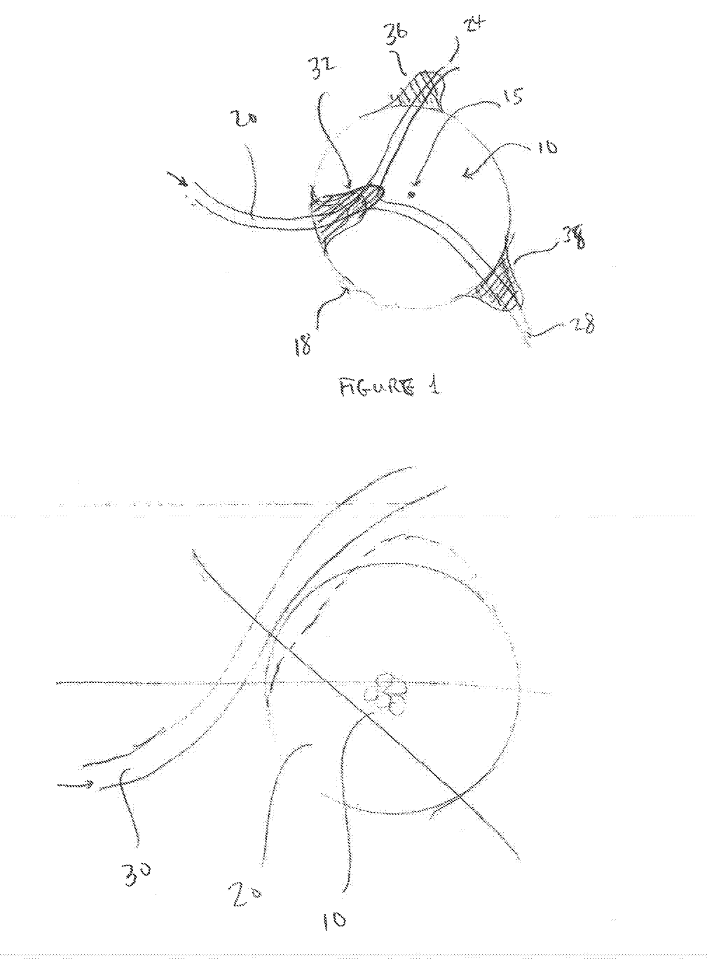

FIG. 1 illustrates the advantage of local gelation of polymers in the circulation that passes through a treatment site. A treatment zone 10 is created by a source of warmth 15, which can be a probe situated below the plane of the drawing, perhaps in another artery or vein. The theoretical outer limit of the treatment zone 10 is, in this example, an essentially circular boundary 18, at which the degree of heating drops below a therapeutic level.

A blood vessel 20 flows through the treatment zone and branches into two smaller vessels 24 and 28. Natural circulation, indicated by small arrows, passes through vessel 20 and out of vessels 24 and 28. However, the blood flow picks up heat from the treatment zone. This causes cooling in the vicinity of the blood entrance into the heating zone, shown as hatched area 32, and causes heating at regions beyond the target zone 10 along the exiting blood vessels, shown as hatched areas 36 and 38. It is likely that tissue in the area 32 will not be properly treated, and that tissue in areas 36 and 38 will be treated even though outside the target zone. This is undesirable. However, if heating is begun, and then followed by instillation of a reverse-gelling poloxamer solution at a location upstream of the target region, leading to vessel 20, then a gel will form in the region being treated. The gel may begin to form in the distal vessels 26 and 28, and once formed, will stop circulation through the treatment site. Then the heat distribution in the zone 10 will more closely approximate the distribution planned for the treatment, having a treatment boundary at the circular border 18. Once heating element 15 is turned off, the tissue will rapidly drop to body temperature by heat transfer through the treated tissue to tissue outside the treatment zone 10. The gelled polymer molecules in the vessels 20, 24, and 28 will become more soluble, and their diffusion away from the gel will increase, resulting in removal of the embolization, so that circulation will resume. The reperfusion of the organ may be accelerated, if desired, by circulation of isotonic fluid at a temperature of less than 37° C., or even less than 30° C., or by other cooling methods as described above. Circulation may be exterior to the organ, and/or through regions of the organ where circulation has not been blocked by gelation of polymer.

If the site needs to be closed after treatment, closure may be attained with any conventional method, including without limitation one or more of sutures, staples, sealant, adhesive, and hemostatic agent, before the reduction of temperature to allow reperfusion of the organ by blood.

### Surgical Removal of Tissues

In addition to thermotherapy, the reversible local embolization technique of the invention is applicable to surgical procedures removing tissue, particularly for removing part of a vascularized or compartmented organ, such as partial removal of liver or kidney. Such highly metabolically active organs require minimization of the anoxia produced by embolization, both spatially and in terms of duration. In such organs, a portion of the tissue adjacent to the site to be surgically removed—for example, a tumor—is subjected to a local warming process. The warming process may include local perfusion, in the normal direction or its reverse, with a warming solution, as well as local heating by other means. Then, when the region adjacent to the region to be excised has been sufficiently warmed, it is perfused with a embolizing solution containing a reverse gelling polymer. The warmth causes rapid local embolization in the heated zone, and little stable embolization outside that zone. Then the tissue to be removed is quickly excised, and a sealing barrier layer is created by conventional means, for example and without limitation by one or more of local cautery, provision of tissue adhesives and barrier materials, and suturing. With proper timing, the rest of the organ can be de-embolized within a few minutes as the applied warming dissipates, and the RGP diffuses away. The dissected and sealed organ can also be cooled immediately to accelerate reperfusion.

### Additional Features

The reverse gelling polymer solution can further comprise other medical materials. These may include, among others, a contrast-enhancing agent, which may be selected from the group consisting of radiopaque materials, paramagnetic materials, heavy atoms, transition metals, lanthanides, actinides, dyes, and radionuclide-containing materials. The solution may further comprises a biologically active agent, which, for example, may comprise one or more of anti-inflammatories, antibiotics, antimicrobials, antivirals, analgesics, antiproliferatives, and chemotherapeutics, or other biologically active agents.

### Equivalents & Incorporation by Reference

All of the patents and publications cited herein are hereby incorporated by reference in jurisdictions permitting the same. Those skilled in the art will recognize, or be able to ascertain using no more than routine experimentation, many equivalents to the specific embodiments of the invention described herein. Such equivalents are intended to be encompassed by the following claims.

1. A method of producing temporary hemostasis in a site in the tissue of a mammal, the method comprising the steps of:
a) introducing into the vasculature of said tissue, at a location leading through the circulation to said site, a temporary embolizing solution comprising a reverse gelling polymer, wherein said embolizing solution has a composition and a concentration which causes it to gel sufficiently at a gel temperature Tg to effectively stop blood flow at said site, said gel temperature Tg being below the local tissue temperature of the tissue being treated;
b) perfusing said site with said reverse gelling polymer composition; and
c) before or during said perfusion, heating said site to a temperature above body temperature;
thereby causing said reverse gelling polymer to gel sufficiently to produce temporary hemostasis at said site of said mammal.

2. The method of claim 1, wherein the local tissue temperature is 37° C. and the gel temperature Tg of said embolizing solution is between about 28° C. and about 36° C.

3. (canceled)

4. (canceled)

5. The method of claim 1, wherein said reverse thermosensitive polymer is a block copolymer, random copolymer, graft copolymer, or branched polymer or copolymer.

6-13. (canceled)

14. A method for performing a surgical procedure at a site in a tissue of a mammal, the method comprising the steps of:
accessing the vasculature providing blood to said site or receiving blood from said site, with a fluid delivery system;
delivering through said fluid delivery system an embolizing solution comprising a reverse gelling polymer that at least partially gels when its temperature rises to a temperature in the range of 28 to 36° C. and below local tissue temperature;
warming said embolizing solution above local tissue temperature at or near said site, thereby firmly gelling the embolizing solution to embolize said site;
maintaining said warming throughout the performance of the surgical procedure, thereby maintaining hemostasis at the site; and
discontinuing the heating at the close of the procedure, thereby allowing the polymer molecules to diffuse away from the gel, thereby causing said gelation to reverse, which allows resumption of blood flow at the site.

15. The method of claim 14, wherein the embolizing solution that gels in the temperature range of 28 to 36° C., and below local tissue temperature, comprises one or more poloxamers or poloxamines as reverse gelling polymers.

16-28. (canceled)

29. A system for thermal treatment of an organ, the system comprising:
means for applying heat to a localized region of an organ, to selectively destroy tissue by heating it to a temperature above 37° C.;
means for locally perfusing said localized region of an organ with an embolizing solution comprising a reverse gelling polymer, wherein the gelling temperature for said reverse gelling polymer is below 37° C.;
whereby reversible local hemostasis is obtained at the site of thermal treatment while heat is applied to said localized region, and said hemostasis spontaneously ceases after the application of said thermal treatment ceases,
wherein the local tissue temperature is 37° C. and the gel temperature Tg of said embolizing solution is between 28° C. and 36° C., and
wherein the reverse gelling polymer is a fractionated polyoxyalkylene block copolymer.

30-33. (canceled)

34. The system of claim 29, wherein the region or site is temporarily embolized by perfusing a larger region of tissue in which said region or site is located with said embolizing solution or composition, but heating only near the region or site, thereby forming a gel in the vicinity of said region or site.

35. The system of claim 29, wherein the local tissue temperature is 37° C. or lower.

36. The system of claim 29, wherein said reverse gelling polymer is a fractionated poloxamer or poloxamine, selected from poloxamers 407, 188, 118, 338 or poloxamines 1107 and 1307.

37. The system of claim 29, wherein said reverse gelling polymer is a fractionated poloxamer or poloxamine.

38. The system of claim 29, wherein said perfusing or infusing begins after the beginning of said heating.

39. The system of claim 29, wherein the heating of the organ is provided by one or more of electromagnetic radiation, sonic energy, heated fluid, a heating pad, a heating element, and heat produced by a surgical tool or instrument.

40. The system of claim 29, wherein said embolizing solution or composition further comprises a contrast-enhancing agent.

41. The system of claim 29, wherein said contrast-enhancing agent is selected from the group consisting of radiopaque materials, paramagnetic materials, heavy atoms, transition metals, lanthanides, actinides, dyes, and radionuclide-containing materials.

42. The system of claim 29, wherein said solution or composition further comprises a biologically active agent, the active agent being selected from the group consisting of anti-inflammatories, antibiotics, antimicrobials, antivirals, analgesics, antiproliferatives, and chemotherapeutics.

43. The system of claim 29, wherein after completion of the procedure, the reperfusion of the organ is accelerated by circulation of isotonic fluid at a temperature of less than 37° C. by one or more route selected from a route that passes through the organ and a route that passes along the exterior of the organ.

44. The system of claim 44, wherein the temperature of the reperfusing fluid is less than 30° C.

45. The system of claim 29, wherein the reverse-gelling polymeric solution gels at a temperature below the body temperature at the site, and the gelation is strengthened by the localized heating of the site above the local body temperature.
